(19) United States
(12) Patent Application Publication
(10) Pub. No.: US 2006/0189922 A1
(43) Pub. Date: Aug. 24, 2006

(54) Dialysis catheter with stiffening member and flow diverting structure

(76) Inventors: Chanaka Amarasinghe, North Brunswick, NJ (US); James Bates, Sparta, NJ (US)

Correspondence Address: Gerard P. Norton;NORTON & DIEHL LLC, Suite 110, 77 Brant Ave., Clark, NJ, 07066, US

(21) Appl. No.: 11/300,170

(22) Filed: Dec. 14, 2005

Related U.S. Application Data
(63) Continuation of application No. PCT/US04/27203, filed on Aug. 21, 2004.
(60) Provisional application No. 60/496,419, filed on Aug. 20, 2003.

(51) Int. Cl. A61M 1/00

(52) U.S. Cl. 604/28; 604/43

(57) ABSTRACT

Disclosed is a double lumen continuous flow dialysis catheter having contiguous lumens of different lengths, the shorter lumen acting as a blood intake lumen and the longer as a blood return lumen. The catheter is designed to ease insertion into the body without the use of a tearaway sheath and to minimize recirculation flow from the blood return lumen to the blood intake lumen and/or prevent the blood intake lumen from becoming compressed against a vessel or body wall during dialysis.

### CROSS-REFERENCE TO RELATED APPLICATIONS

This application is a continuation of International Application No. PCT/US2004/027203, filed on Aug. 20, 2004, and incorporates by reference and claims priority to International Application No. PCT/US2004/027203, filed on Aug. 20, 2004, and U.S. Provisional Patent Application No. 60/496,410, filed on Aug. 20, 2003.

### BACKGROUND

This invention relates to a catheter primarily for use in dialysis and more specifically, to a dialysis catheter having a double lumen, a diverting structure and a temporary stiffener.

Dialysis is currently performed in two basic ways. First, the conventional way employing two needles, one for removing the blood from the vein or body for processing in a dialysis unit and the other needle for returning processed blood back into the vein or body. In this conventional technique for dialysis, the two needles must be spaced apart a sufficient distance so as to prevent the cleansed blood from re-entering the blood outlet needle and returning to the dialysis unit but must be sufficiently close to each other to prevent the vein from collapsing.

A second known manner of performing dialysis utilizes a single needle in which blood is both extracted and returned through the same needle. However, single needle dialysis requires an intermittent occlusion machine which is capable of the cyclical operation necessitated by the single lumen needle with bi-directional flow. In addition, single needle dialysis can only operate within limited flow rates and accordingly is not suitable for all patients.

For repeated dialysis requirements, a method utilizing two long tubes of almost unequal length attached side by side is also known. In general, the tubes are introduced into the jugular vein and remain there for several days, weeks or even months, during which hemodialysis is performed.

Also known in the art as shown in U.S. Pat. No. 4,134,402 is to provide a double lumen catheter for dialysis capable of achieving blood flow rates comparable to the conventional two needle system while requiring only one puncture.

Further known in the art are multiple lumen catheters capable of use with a conventional dialysis unit as disclosed in U.S. Pat. Nos. 5,221,255, 5,221,256 and 5,486,159.

### SUMMARY

Briefly stated, the present invention consists of a single catheter having two contiguous lumens or conduits, one slightly longer than the other, containing a temporary stiffening member, designed to ease insertion of the catheter into the body without the use of a tearaway sheath. The catheter is generally inserted into the vein or body in the direction of blood flow. The shorter lumen then serves as a blood intake lumen and the longer lumen, the end of which is positioned away from the end of the shorter lumen in the direction of blood flow, serves as a blood return lumen. A distally located diverting structure is also provided to ensure that cleansed blood returning to the vein or body will not re-enter the intake lumen, but rather will be carried “downstream”. The diverting structure also functions to prevent the blood intake lumen from becoming compressed against the vessel or body wall during dialysis.

### BRIEF DESCRIPTION OF THE DRAWINGS

FIG. 1 is an illustration of a double lumen catheter of the present invention;

FIG. 2 is a cross section of the double lumen catheter of FIG. 1;

FIG. 3 is an illustration of the front distal section of the double lumen catheter of FIG. 1;

FIG. 4 is an illustration of the rear proximal section of the double lumen catheter of FIG. 1;

FIG. 5 is an illustration of another embodiment of the front distal section of a double lumen catheter of the present invention;

FIG. 6 is an illustration of a further embodiment of the front distal section of a double lumen catheter of the present invention;

FIG. 7 is an illustration of still another embodiment of the front distal section of a double lumen catheter of the present invention;

FIG. 8 is an illustration of another embodiment of the distal tip section of a double lumen catheter of the present invention;

FIG. 9 is an illustration of still another embodiment of the distal tip section of a double lumen catheter of the present invention; and

FIG. 10 is a schematic showing blood flow during dialysis.

### DETAILED DESCRIPTION

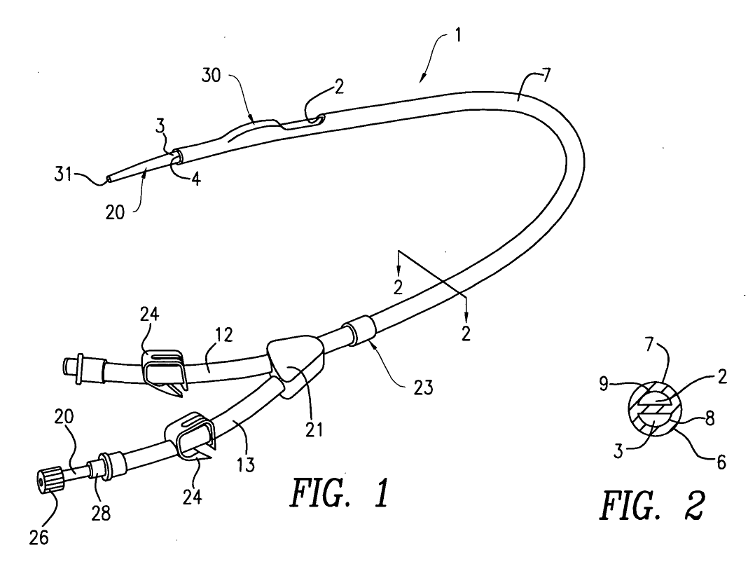

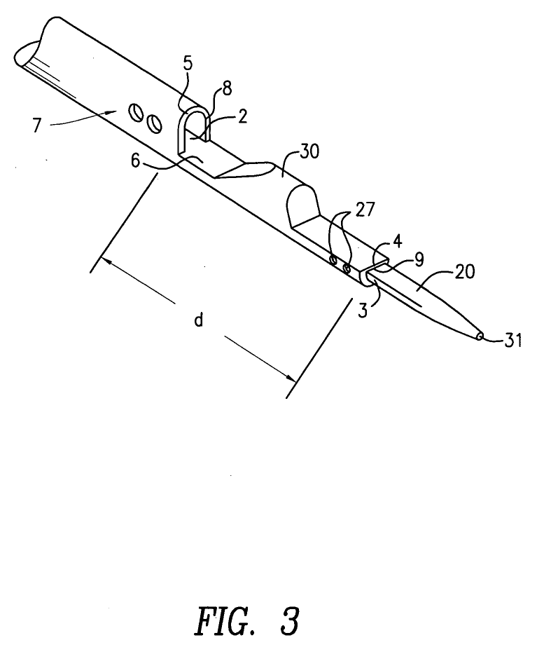

Referring to FIGS. 1 and 2, it can be seen that the present invention consists of a single catheter 1 having intake lumen 2 and return lumen 3 terminating in blood intake aperture 8 and blood return aperture 9, respectively. The term “catheter” as used in this specification includes rigid metal devices such as needles as well as flexible plastic devices such as cannula. As illustrated in FIGS. 1, 3, 5 and 7, blood return aperture 9 at the end of return lumen 3 extends distally beyond blood intake aperture 8 at the end of intake lumen 2 a sufficient distance to prevent mixing of the blood during the hemodialysis operation. The precise distance by which return lumen 3 extends distally beyond intake lumen 2 is determined by the rate of blood flow, the angle of entrance of the double lumen, and the size of the vein or vessel in which the blood is flowing. Strictly by way of example, for an average vessel, (e.g., a surgically constructed blood vessel with rapid blood flow rates) having a diameter of ½ inch and processing blood at approximately 500 cubic centimeters per minute, the separation distance “d” (see FIG. 3) between return lumen 3 and intake lumen 2 would be approximately ¼ inch. This separation could be as large as ¾ inch or even larger in some circumstances. In a preferred embodiment, the distal end of each lumen may be provided with beveled edges 4 and 5 sloping outwardly and away from the distal catheter tip to promote ease of insertion of catheter 1.

A diverting structure 30 extending outward from septum wall 6 and located distally of blood intake aperture 8 as shown in FIG. 3 can also be provided to function as a flow diverter to reduce access recirculation and to raise fluid pressure in the vicinity of blood intake aperture 8. Diverting structure 30 can also function to prevent intake lumen 2 from becoming compressed against the vessel or body wall during dialysis. Diverting structure 30 can be any shape or form so long as it diverts recirculation flow from blood return aperture 9 away from blood intake aperture 8 and/or prevents intake intake lumen 2 from becoming compressed against the vessel or body wall during dialysis. As shown in FIGS. 1 and 3, diverting structure 30 is in the form of a frustum and completes a phantom outline of intake lumen 2 projected in a proximal direction from blood intake aperture 8. Further, as depicted in FIGS. 1, 3, 5 and 7, diverting structure 30 has a slanted face opposed to recirculation flow to minimize catheter insertion trauma. Preferably, diverting structure 30 is made from a material of heavier construction than that which forms outer wall 7 of catheter 1, such that it also functions as a tissue dilator to ease insertion of catheter 1. Alternatively, diverting structure 30 can be made of the same material as outer wall 7.

Referring to FIG. 2, it can be seen that in the region where the lumens are contiguous, the lumens are separated by septum wall 6 such that intake lumen 2 and return lumen 3 are each of “D” shaped cross section. At least a portion of return lumen 3 that extends distally beyond intake lumen 2 is preferably of circular cross section (FIG. 9). Septum wall 6 can be relatively thin construction inasmuch as its only function is to separate the blood return conduit 8 from the blood intake conduit 9. Outer wall 7, in contrast, must serve as a supporting wall and accordingly may be thicker than septum wall 6.

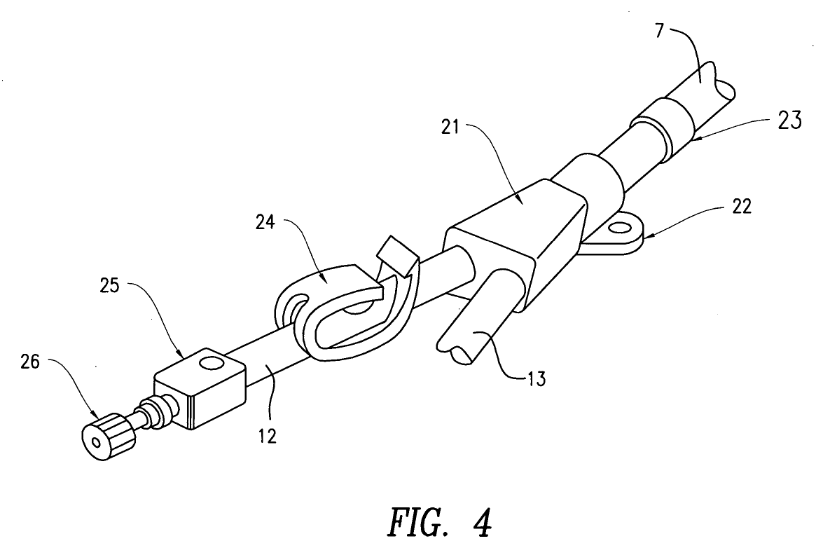

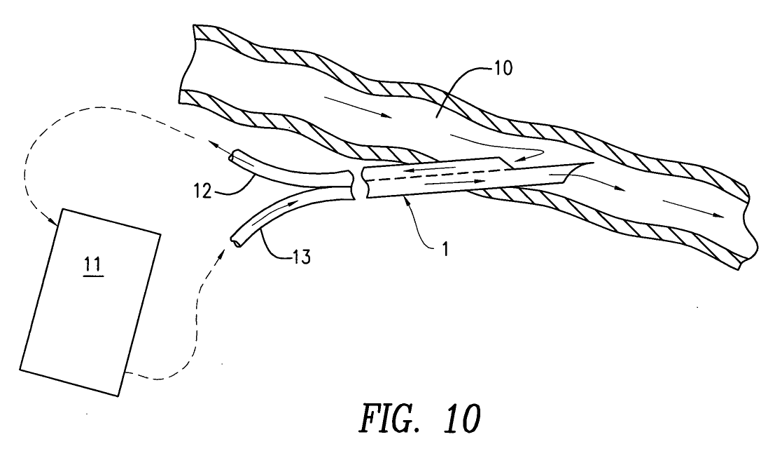

Referring to FIG. 10, the actual operation will be described with reference to a double lumen catheter constructed in accordance with the present invention. The double lumen catheter 1 is inserted into the vein or body 10 in the direction of blood flow. The noninserted ends of the lumens are connected to a dialysis unit 11. This connection can be accomplished by separating contiguous lumens 2 and 3 into two noncontiguous connector tubes 12 and 13 (FIG. 1) of circular cross section with standard luer ends so that conventional coupling members may be utilized. The point of separation can be included in a housing 21 to form a conventional hub (see FIGS. 1 and 4). With dialysis unit 11 in operation, blood flows from the vein or body into intake lumen 2 through connector tube 12 to dialysis unit 11 where blood is processed. The blood is then returned to the vein or body through connector tube 13 and out of return lumen 3. The distal tip of catheter 1 can also include one or more side openings or ports 27 formed through outer wall 7 in fluid communication with return lumen 3 (FIG. 3), also functioning to return blood to the patient's body. The returning blood enters the vein or body at a point displaced some distance away from the point where blood enters intake lumen and in the direction of blood flow in the vein or body. The blood flow through the body or vein then carries this processed blood away from intake lumen 2. As shown in FIGS. 1 and 4, catheter 1 can be provided with standard hardware such as rotatable suture ring 22 and fabric (e.g., polyester felt) cuff 23, while connector tubes 12 and 13 can be provided with standard hardware such as tube clamps 2-4, printed (e.g., product name, priming volume, etc.) ID tags on hubs 25 and luer caps 26, the use of such hardware being known in the art.

In order to aid in insertion of catheter 1, usually by the Seldinger technique, and navigation through small vessels, a stiffening member 20 (FIGS. 1 and 3) may be provided in conjunction with beveled edges 4 and 5 and diverting structure 30. Stiffening member 20 is preferably inserted into the proximal end of connector tube 13 connected to return lumen 3. Once positioned, the distal end of stiffening member 20 extends distally of blood return aperture 8 at the distal tip of catheter 1. A luer lock 27 is provided at the proximal end of stiffening member 20 to secure it to threads 28 at the proximal end of connector tube 13 during insertion of catheter 1. Stiffening member 20 preferably has an internal lumen 31 extending therethrough for receiving a guidewire for proper placement of catheter 1. Prior to operation of dialysis unit 11, luer lock 27 is unscrewed from proximal threads 28 of connector tube 14, allowing removal of stiffening member 20 from catheter 1.

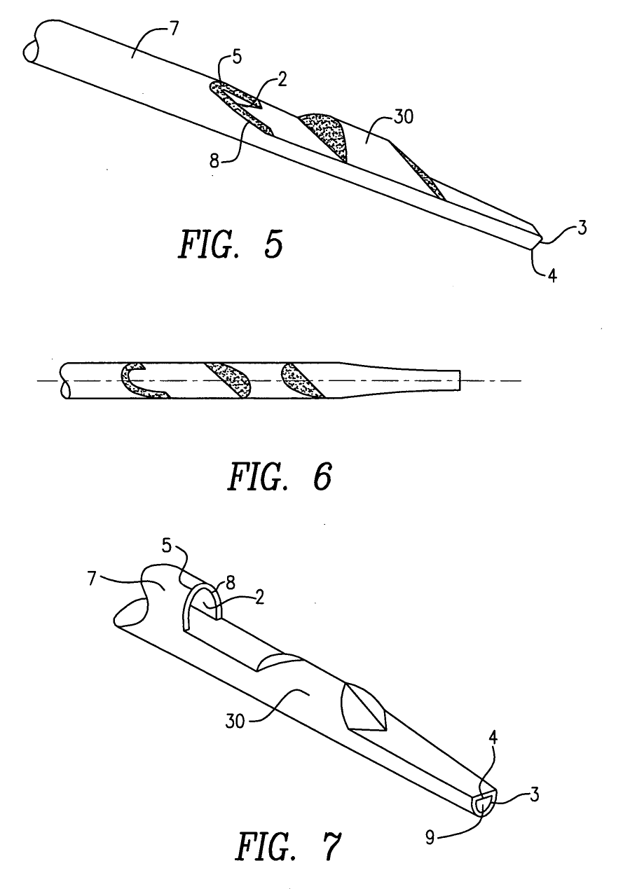

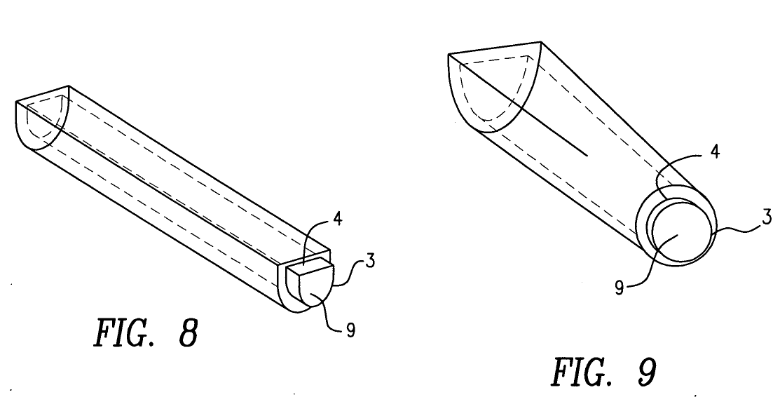

From the foregoing, the present invention has been sufficiently described to enable others skilled in the art, by applying current knowledge, to adapt the same for varying conditions of use without departing from the essential items of novelty involved, which are intended to be defined and secured by claims to this application. Some of those adaptions are shown in the additional embodiments depicted in FIGS. 5, 6, 7, 8 and 9.

1. A double lumen catheter, comprising:
an elongated tube having a unitary outer wall and longitudinal septum wall dividing an interior of said tube into an intake lumen and a return lumen, said intake lumen extending from a proximal end of said tube to an end terminating in a distal intake aperture, said return lumen extending substantially contiguously with said intake lumen from said proximal end of said tube to an end terminating in a distal return aperture spaced distally forward of said intake aperture;
a diverting structure extending outward from said outer wall of said longitudinal septum wall and positioned distally forward of said intake aperture; and
a stiffening member removably positionable within said catheter tube.

2. The double lumen catheter of claim 1, wherein the distal ends of the intake lumen and the return lumen are beveled.

3. The double lumen catheter of claim 1, wherein the diverting structure has a slanted face opposed to recirculation.

4. The double lumen catheter of claim 1, wherein the diverting structure comprises a material of heavier construction than the outer wall of said catheter.

5. The double lumen catheter of claim 1, wherein the diverting structure is made of the same material as the outer wall of said catheter.

6. The double lumen catheter of claim 1, wherein the diverting structure is in the form of a frustum and completes a phantom outline of said intake lumen projected in a proximal direction from said intake aperture.

7. The double lumen catheter of claim 1, wherein the diverting structure functions to prevent said intake lumen from becoming compressed against a vessel or body wall during dialysis.

8. The double lumen catheter of claim 1, wherein the contiguous portions of the intake lumen and the return lumen are each of “D” shaped cross section.

9. The double lumen catheter of claim 1, wherein at least a portion of the return lumen that extends distally of the intake lumen is of circular cross section.

10. The double lumen catheter of claim 1, wherein said outer wall is thicker than said septum wall.

11. The double lumen catheter of claim 1, further comprising two noncontiguous connector tubes for attachment of said catheter to a dialysis unit.

12. The double lumen catheter of claim 1, wherein the outer wall of said catheter further comprises one or more side openings in fluid communication with the return lumen.

13. The double lumen catheter of claim 1, wherein said return aperture is spaced distally forward of said intake aperture a distance of greater than ¾ inch.

14. The double lumen catheter of claim 1, wherein the stiffening member is removably positionable within the return lumen.

15. The double lumen catheter of claim 1, wherein the stiffening member has an internal lumen extending therethrough for receiving a guidewire.

16. A dialysis system, comprising:
a dialysis unit; and
the double lumen catheter of claim 1 connected to said dialysis unit.

17. A method for performing dialysis in a subject, comprising:
withdrawing blood from a vein of said subject through the intake aperture of the intake lumen of the double lumen catheter of claim 1;
passing said blood through a dialysis unit; and
returning said blood to said vein through the return aperture of the return lumen of said double lumen catheter.
